(19) United States
(12) Patent Application Publication
(10) Pub. No.: US 2012/0328544 A1
(43) Pub. Date: Dec. 27, 2012

(54) Dermatological Treatment Methods And Formulations

(75) Inventors: Richard F. Stockel, Bridgewater, NJ (US); Anthony J. Sawyer, Albuquerque, NM (US); Anthony E. Winston, East Brunswick, NJ (US)

(73) Assignee: Nevada Naturals, Inc., Albuquerque, NM (US)

(21) Appl. No.: 13/600,935

(22) Filed: Aug. 31, 2012

Related U.S. Application Data
(63) Continuation-in-part of application No. 12/583,919, filed on Aug. 27, 2009, now abandoned.
Application No. 12/583,919 is a continuation-in-part of application No. 11/637,450, filed on Dec. 12, 2006, now abandoned.
Application No. 11/637,450 is a continuation-in-part of application No. 10/741,346, filed on Dec. 22, 2003, now abandoned.
(60) Provisional application No. 61/196,455, filed on Oct. 17, 2008.

(51) Int. Cl. A61K 31/14; A61K 31/223; A61K 8/92; A61Q 3/00; A61P 17/00; A61P 17/10; A61Q 19/00; A61Q 15/00; A61K 31/60; A61P 31/04

(52) U.S. Cl. 424/61; 514/162; 514/551; 514/643; 424/65

(57) ABSTRACT

A dermatological composition for application to the skin or nails comprises a salt of a cation and an anion. The cation is derived from a monomeric or polymeric molecule that will generate an amidine moiety, a guanidine moiety or a biguanide moiety. The anion is derived from a monomeric or polymeric molecule that will generate a carboxylic acid moiety. The composition may be prepared by a metathesis or acid-base reaction.

### REFERENCE TO RELATED APPLICATIONS

This application is a continuation-in-part of application Ser. No. 12/583,919, filed Aug. 27, 2009, which was filed as a continuation-in-part of application Ser. No. 11/637,450, filed Dec. 12, 2006, which in turn was filed as a continuation-in-part of application Ser. No. 10/741,346, filed Dec. 22, 2003 (now abandoned). Ser. No. 12/583,919 is also a continuation-in-part of application Ser. No. 61/196,455, filed Oct. 17, 2008.

### FIELD OF THE INVENTION

The invention relates to the dermatological compositions containing biocidal salts providing exceptional antimicrobial, antibacterial, antiviral and/or antifungal activity and reduced undesirable side effects such as skin irritation and can be used for the treatment of mammalian skin, and nail disorders. The unique biocidal salts of the invention have been found to be extremely useful for the treatment of many skin conditions such as, but not limited to, ichthyosis, eczema, dry skin psoriasis, pruritus, plantar hyperkeratosis, acne, keratosis, herpes virus, skin blemishes, warts, and other skin conditions resulting from but not limited to bacteria, viruses and fungal infections of the skin etc.

The invention provides compositions and methods for the alleviation of both visible and non-visible, i.e., pre-emergent, dermatological lesions associated with changes in normal keratinization, cutaneous infection, epidermal formation or pilosebaceous function such as acne, psoriasis, seborrhea, ingrown and pseudofolliculitis barbae, and hyper-pigmented skin.

The invention significantly expands the options for treatment of dermatological conditions by allowing the medical professional to choose from a plethora of biocidal agents having the proper chemical characteristics required to prepare a composition to be used for a specific skin condition. For example, a cationic (or conjugate base) molecule with antimicrobial, antibacterial, antiviral or antifungal properties can be combined with a selected anionic (or conjugate acid) molecule to provide the desired therapeutic outcome as well as a benefit to the skin.

The use of environmentally-beneficial materials, especially those that are referred to as “green” is an important consideration in selecting a topically-applied or ingested composition. The use of natural or naturally-derived materials is also of significant interest for topically-applied or ingested compositions. Accordingly, the use of both green and naturally-derived materials in a composition having exceptional antimicrobial, antibacterial, antiviral and/or skin-beneficial properties would be clearly desirable. If all of the components of the compositions of the invention are GRAS (Generally Regarded As Safe) and are approved for food use, the resulting compositions could also be ingested with little or no side effects.

In respect to the term “dermatological” used throughout the specification and claims, it should be understood that the compositions of the invention are useful not only for the treatment of mammalian skin, but also for the treatment of treatment of mammalian nails.

### BACKGROUND OF THE INVENTION

The prior art is replete with various approaches to the treatment of dermatological conditions. Benzoyl peroxide is well known as a medicament for the treatment of acne. Often the benzoyl peroxide is combined with an antibacterial agent or an antibiotic to extend its spectrum of activity as disclosed in, e.g., U.S. Pat. No. 5,767,098. However there are several disadvantages to this combination approach. With prolonged usage that is typically required for the treatment of acne, the bacterial flora becomes resistant thus rendering the antibacterial agent or the antibiotic less effective in subsequent treatment. Moreover, the benzoyl peroxide component of the combination is oxidatively unstable.

Numerous publications and patents disclose the use of alpha.-hydroxy acids for the treatment of dermatological conditions. For example, U.S. Pat. No. 4,363,815 discloses the use of such compounds for the treatment of dry skin, ichthyosis, plantar hyperkeratosis, Darier's disease, keratoses, acne, psoriasis, eczema, pruritis, warts and herpes virus. Other patents describe the use of various incipients to lessen skin irritation and stinging, e.g., lactate salts, amphoteric salts (see U.S. Pat. No. 5,420,106), ascorbic acid derivatives (U.S. Pat. No. 5,703,122), amino salts (see Cosmetics and Toiletries, volume 113, March 1998, p. 55). However alpha-hydroxyl acids and the above mentioned incipients are not primarily used to cure skin infections but to remove dead keratin.

Salicylic acid is frequently disclosed as an active ingredient for the treatment of a wide variety of skin conditions, e.g., psoriasis, skin atrophy, skin wrinkles, acne, etc., see U.S. Pat. Nos. 5,776,920; 5,780,457; 5,780,458; 6,436,417. In all of these cases, salicylic acid must be modified with other incipients to prevent undesirable side reactions such as skin irritation and the like.

There is a need for a safe and efficacious course of treatment for severe acne, especially that which gets at the root cause of skin problems due to bacterial infections. Antibiotics such are sometimes prescribed for treating acne. However, there are potential side effects to antibiotic treatment including subsequent resistance to the effectiveness of antibiotics when needed to treat this and other diseases. The only treatment to date which has proven to be effective for non-bacterial infections is isotretinoin which is orally administered. This medication has many undesirable side effects including the possibility of causing birth defects when administered to pregnant women.

### SUMMARY OF THE INVENTION

This invention pertains to a novel method of using the dermatological compositions of this invention. The compositions comprise a salt of a monomeric or polymeric cation and a monomeric or polymeric carboxylate anion. This invention also pertains to dermatological formulations for treating mammalian skin, or nails comprising a salt of a monomeric or polymeric cation and a carboxylate anion. The dermatological salts of the invention preferably are strongly adherent to the surface of the skin. The cation of the salt may be an amidinium, a guanidinium, or a biguanidinium, or a quaternary ammonium cation, while the anion may be a carboxylate. The cation may also be an amine acid salt of an azole. The cation may also be a cation of an antibiotic with an amine functional group. Such compositions possess antimicrobial, antibacterial, antifungal, and antiviral properties. In addition they may have mammalian skin or nail-beneficial properties.

### DETAILED DESCRIPTION OF THE INVENTION

The formulations of this invention comprise from about 0.02 wt % to 5 wt % of a dermatological salt of a monomeric or polymeric cation and a monomeric or polymeric carboxylate anion such salt having an aqueous solubility at room temperature of less than 5 wt %, preferably equal to or less than 2 wt % and greater than about 0.01 wt % and at least one or more of the following: (1) from about 0.05% to about 20 wt % of a water absorbing hydrophilic polymer with a molecular weight above 2000, (2) from about 1% to about 90 wt % of an anionic, nonionic or amphoteric surfactant or soap, (3) from about 0.02 to about 25 wt % of an emollient (4) from about 0.02 to about 99 wt %, preferably up to 50 wt % of an emulsifier, (5) buffers to provide a pH between about 3.0 and 7.0, (6) L-cysteine or L-N-acetyl cysteine, and (7) from about 2% to about 95 wt % moisture. Other ingredients can be added to formulations of the invention. These include but are not limited to a suspending agent, a carrier liquid, moisturizers, humectants, an antiperspirant active, sunscreen agents, vitamins, dyes, fragrances, preservatives, antioxidants, detackifying agents, thickening agents, processing aids, preservatives chelating agents, pH adjusters/buffering agents, foam stabilizers, opacifiers and similar types of compounds.

The formulation supplies a dermatological salt with limited water solubility. This salt partially dissolves in water to deliver an effective quantity of an antimicrobial ingredient and also a skin or nail benefit agent for an extended period. The limited solubility salt ensures the continuing presence of an effective amount of antimicrobial active(s). It should be noted that an important aspect of the technology is that the controlled-release salt is actually a single compound that does not require encapsulation to achieve its controlled release characteristics. As the compound dissolves, it releases species which are active against both bacteria and fungi. The controlled release characteristics of the salt are dependent on its solubility. The controlled release salt thus provides a reservoir of active antimicrobial ions to be released as the dissolved ions are used up or depleted. Additionally, the formulations of this invention can contain the active antimicrobial agent that is included in the controlled release salt. As that agent is depleted, the salt can release additional active cations and/or anions contained in the salt to provide antimicrobial activity over a prolonged period.

The present invention also provides a method of treating the skin, or nails by applying formulations which provide extended release antimicrobial, antibacterial, antiviral or antifungal properties as well as long lasting benefits. The method of this invention comprises applying a formulation to the skin, or nails comprising a dermatological salt, whereby said salt releases skin benefit agents as well as antimicrobial, antibacterial, antiviral or antifungal ions when it partially dissolves in sweat or other moisture present on the skin, leaving residual undissolved salt on the skin as a reservoir for subsequent dissolution and release of cations and anions, as the original dissolved ions are used up or depleted. The presence of undissolved salt results from the low solubility of the salts. The amounts of dissolved and undissolved salt will depend on the solubility of the dermatological salt, the amount of applied dermatological salt and the amount of moisture present on the skin. The required aqueous solubility of the dermatological salt will vary depending on the end use of the formulation of the invention. Where the composition is used with an abundant quantity of water, e.g. for a body wash used in the shower or for an ointment treatment which needs to remain on the skin for a few days despite showering, there is a need to more severely restrict the solubility to ensure the retention of undissolved dermatological salt on the skin after product use. In such a case the solubility of the dermatological salt should preferably be less than 0.5% or even lower. Where the supply of water is more limited, e.g. for an ointment which is meant to be retained on the skin for a about day between showers or baths, a solubility of less than 2% or even 5% might be preferable. On the other hand the dermatological salt should have a solubility of greater than about 0.01% to ensure that sufficient cations and anions are released to provide their desired effects.

In addition to the dermatological salt, formulations of the invention may comprise hydrophilic humectant polymers. These materials absorb and/or retain moisture on the skin to assure the presence of an aqueous medium for salt dissolution and hence release of its desired ions. Furthermore one or more surfactants can help clean the skin as well as retain surface moisture on the skin surface. An emollient glycerol ester of a fatty acid, if utilized, is beneficial in smoothing the skin and reducing irritation. A preferred glycerol ester of a fatty acid is a glycerol ester of a C₈-C₁₄ fatty acid. The presence of buffers assures the moisture on the skin has a non-irritating pH and can be important in promoting the efficacy of the ions released from the dermatological salt. In a preferred embodiment of the invention natural or naturally derived ingredients are used and the extended release, skin adherent salt is derived from a natural amino acid and fatty alcohols or fatty acids from renewable sources.

The method of this invention comprises the application of a formulation to the skin, or nails comprising from about 0.02 wt % to 5 wt % of a salt of a monomeric or polymeric cation and a monomeric or polymeric carboxylate anion with a solubility of less than 5 wt %, preferably equal to or less than 2 wt % and greater than about 0.01 wt % and at least one or more of the following: (1) from about 0.05% to about 20 wt % of a water absorbing hydrophilic polymer with a molecular weight above 2000, (2) from about 1% to about 90 wt % of an anionic, nonionic or amphoteric surfactant or soap, (3) from about 0.02 to about 25wt % of an emollient, (4) from about 0.02 to about 100 wt % of an emulsifier, (5) buffers to provide a pH between about 3.0 and 7.0, (6) L-cysteine or L-N-acetyl cysteine, and (7) from about 2% to about 95 wt % moisture.

The present invention provides a method of treating the skin or nails by applying formulations which provide extended release benefits. The method of this invention comprises applying a formulation comprising an extended-release, undissociated, adherent salt, which releases antimicrobial, antibacterial, antiviral or antifungal cations and skin or nails benefit anionic agents when it partially dissolves in moisture present on the skin or nails. In addition, formulations of the invention may comprise hydrophilic humectant polymers, one or more surfactants, an emollient glycerol ester of a fatty acid, a buffer or water. In a preferred embodiment of the invention natural or naturally derived ingredients are used and the extended release, skin or nail adherent salt is derived from a natural amino acid and fatty alcohols or fatty acids from renewable sources.

### The Salts of the Invention

The dermatological salts employed in the formulations and methods of the invention may be formed by any means such as by a metathesis or an acid-base reaction. In the case of either type of reaction, a monomeric or polymeric cationic molecule is reacted with a monomeric or polymeric anionic molecule. For the metathesis reaction, the cationic molecule is chosen such that the resultant salt will contain a cation of an amidine, a cation of a guanidine, a cation of a biguanide, a cation of an azole with an amine functional group, a cation of an antibiotic with an amine functional group, or a quaternary ammonium cation while carboxylate anion is chosen to form the salt. For the acid-base reaction, the cation will be present in the form of a free base such that the resultant salt will contain a cation that may be an amidinium, a guanidinium, a biguanidium, a cation of an azole with an amine functional group, a cation of antibiotic with an amine functional group, or quaternary ammonium cation and the anion will be a carboxylate capable of protonating the free base thereby resulting in a salt that will contain a carboxylate ion. Another guanidine of this invention is polyhexamethylene guanidine.

As mentioned above, the monomeric or polymeric cations include guanidinium, amidinium, biguanidium, a cation of an azole with an amine functional group, a cation of an antibiotic with an amine function group, and quaternary ammonium cations. Such materials possess superior antimicrobial activity and may be characterized as having two or three nitrogen atoms attached to a carbon atom that will readily accept a proton to form a protonated imino functionality. The driving force for this to occur is due to resonance in forming an energetically stabilized protonated imino group in the electronic ground state. Furthermore, each of the functionalities from a monoacid base with pKₐ from about 7.5 to about 13, because by accepting a proton, they form a symmetrical cation that is stabilized by delocalization. Such antimicrobial compounds may be monomeric or polymeric and may contain functional groups such as aliphatic, aromatic or alicyclic groups. The cidal mechanism for all of these functionalities is the same.

Suitable examples of cationic molecules that will result in a cationic amidine moiety are propamidine and dibromopropamidine. Suitable examples of cationic molecules that will result in a cationic biguanide are chlorhexidine, hexetidine, alexidine and polyhexamethylene biguanide hydrochloride. Suitable cationic quaternium ammonium cations are benzalkonium and benzethonium. Suitable azoles include cloconazole, clotrimazole, cyproconazole, ketoconazole, fenbucanozole, miconazole, myclobutanil, propiconazole, tebuconazole, and triadimefon. Suitable antibiotics include clinafloxacin, clindamycin, doxycycline, erythromycin, lincomycin, minocycline, tazarotene and tetracycline

Particularly useful cationic biocides are the N^{α}-(C₈-C₁₄) alkanoyl dibasic amino acid ethyl ester salts. Preferably, the di-basic amino acid is selected from the group consisting of arginine, lysine, and histidine and ornithine. Preferably, the cationic molecule comprises N^{α}-lauroyl-L-arginine ethyl ester which is typically employed as the hydrochloride salt (N^{α} lauroyl-L-arginine ethyl ester is also referred to herein below as “LAE”). Other suitable examples of cationic molecules are N^{α}-lauroyl-L-histidine ethyl ester and N^{α}-lauroyl-L-lysine ethyl ester. The dibasic amino acid cation is selected from the group consisting of N^{α}-lauroyl-L-arginine ethyl ester, N^{α}-lauroyl-L-histidine ethyl ester and N^{α}-lauroyl-L-lysine ethyl ester.

As mentioned above, the anionic portion of the dermatological salt is chosen such that the salt will contain a monomeric or polymeric carboxylate. Such carboxylates may be obtained from any monomeric or polymeric, aliphatic, alicyclic or aromatic saturated or unsaturated carboxylic acid

Examples of useful carboxylate ions for the dermatological salt are those derived from lauric acid, palmitic acid, myristic acid, oleic acid, stearic acid, dehydroacetic acid and undecylenic acid. Also useful are those derived from a monobasic aromatic carboxylic acid molecule that contains a phenol group such as mandelic acid or salicylic acid (which is preferred).

For many dermatological applications, it is preferred that the monobasic aliphatic or aromatic carboxylate contain a hydroxy group, e.g. an alpha-hydroxy group or a beta-hydroxy group or a ketone group. Those derived from monobasic aliphatic carboxylic acids containing an alpha-hydroxy group are most preferred. Suitable examples of such carboxylic acids are glycolic acid, gluconic acid, glyceric acid and lactic acid.

Carboxylates can also be derived from an aliphatic or aromatic carboxylic acid in which at least two carboxylic acid groups are present. Suitable examples of such acids are citric acid, malic acid, tartaric acid, glutaric acid, glutamic acid, azelaic acid, and their derivatives.

If a composition included GRAS ingredients that have an antimicrobial cation as well as a naturally-occurring anion that provides skin benefits, then such approach would be beneficial in reducing potential irritation from harsh chemicals while improving skin health. essential fatty acids, e.g., omega acids including 3,6,9 types, etc.; alpha-hydroxy acids and their derivatives (esters and salts), e.g., alpha-hydroxyacetic acid (also known as glycolic acid), alpha-hydroxypropionic acid (also known as lactic acid), alpha-hydroxytetranoic acid, alpha-hydroxyhexanoic acid, alpha-hydroxyoctanoic acid (also known as alpha-hydroxy-caprylic acid), alpha-hydroxynonanoic acid, alpha-hydroxydecanoic acid, alpha-hydroxy-undecanoic acid, alpha-hydroxydodecanoic acid (also known as alpha-hydroxylauric acid), alpha-hydroxytetradecanoic acid, alpha-hydroxyhexadecanoic acid, alpha-hydroxy-octadecanoic acid, alpha-hydroxyoctaeicosanoic acid, etc.; beta-hydroxy acids and their derivatives (esters and salts), e.g., salicylic acid, etc.; certain amino acids, e.g. L-cysteine anion or L-N-acetyl cysteine. Any hydroxy acid that alleviates the symptoms of an undesirable skin condition may be used. Accordingly, the hydroxyl acid may be an alpha, beta, gamma, delta, epsilon or omega hydroxyl acid. L-cysteine and L-N-acetyl cysteine can also be used in particular for aiding the penetration of antimicrobial or antifungal actives into the nails as either anions attached to a cation of this invention or as an additive with the salts of this invention. If the antimicrobial component of the composition is considered “Green and Naturally Derived”, the resultant composition would be preferable to those compositions that utilize natural materials to treat skin conditions.

It is preferred that the cationic and anionic molecules be chosen such that the resultant salt will exhibit a maximum solubility in aqueous media of about 5 wt. %, preferably 2 wt. %. Such salts with limited solubility provide extended-release of the cationic biocidal ions and the anionic skin benefit ions and generally increase adherence to the skin.

Especially preferred salts of the invention are the laurate of N^{α}-lauroyl-L-arginine ethyl ester, the salicylate of N^{α}-lauroyl-L-arginine ethyl ester, the lactate of N^{α}-lauroyl-L-arginine ethyl ester, the citrate of N^{α}-lauroyl-L-arginine ethyl ester, the malate of N^{α}-lauroyl-L-arginine ethyl ester, the gluconate of N^{α}-lauroyl-L-arginine ethyl ester, the azelate of N^{α}-lauroyl-L-arginine ethyl ester, the glycolate of N^{α}-lauroyl-L-arginine ethyl ester, the glycerate of N^{α}-lauroyl-L-arginine ethyl ester, the hyaluronate of N^{α}-lauroyl-L-arginine ethyl ester, the arachidonate of N^{α}-lauroyl-L-arginine ethyl ester, the oleate of N^{α}-lauroyl-L-arginine ethyl ester (C₁₈, unsaturated), the linoleate of N^{α}-lauroyl-L-arginine ethyl ester (C₁₈, polyunsaturated), the a-linoleate of N^{α}-lauroyl-L-arginine ethyl ester acid (ALA), the eicosapentaenoate of N^{α}-lauroyl-L-arginine ethyl ester acid (EPA), the docosahexaeonate of N^{α}-lauroyl-L-arginine ethyl ester (DHA), the erucate of N^{α}-lauroyl-L-arginine ethyl ester, the tartrate of N^{α}-lauroyl-L-arginine ethyl ester and the 3-hydroxypropionate of N^{α}-lauroyl-L-arginine ethyl ester.

The salts of the invention may be prepared either prior to inclusion into a specific dermatological composition, but also as an in-situ reaction while preparing the dermatological composition For many, but most certainly not all, applications, the salts of the invention will be contained in compositions in the form of emulsions, nano-emulsions, micro-emulsions, gels, creams, dispersions, suspensions, foams, sprays, etc.

The salts of the invention have been found to be extremely effective against a wide variety of microorganisms, e.g., bacteria and fungi.

### Formation of Emulsions of the Salts of the Invention

As mentioned above, the salts of the invention have limited water solubility. Therefore, for many dermatological applications, it is desirable to utilize the salts in the form of emulsions, nano-emulsions or micro-emulsions. The following is a generalized procedure for preparing emulsions, nano-emulsions or micro-emulsions of the salts. However, alternative methods can also be employed.

First, the salt of the invention is dissolved in the minimum amount of a solvent that will completely dissolve the selected salt in the amount that is intended for use in the desired dermatological medicament. The solvent of choice will be one with the appropriate Hildebrand solubility parameter. The solubility parameter is a numerical value that indicates the relative solvency behavior of a specific solvent. Hildebrand solubility parameters of about 8.5 to about 22.0 are generally suitable for solubilization of the salts. Exemplary solvents with the requisite Hildebrand solubility parameters include ethanol, glycerin, propylene glycol, sorbitol, methanol and the like.

The desirable Hildebrand solubility parameter will depend on the ionic/covalent bonding energies of the salts of the invention. The correct solvent will be one having a relatively low Hildebrand solubility parameter if the bonding has more covalency and a relatively high Hildebrand solubility parameter if the bonding is more ionic. Of course, combinations of correct solvents may also be utilized to dissolve the salts of the invention.

Thereafter, a surfactant is added to the dissolved salt. The surfactant may be cationic, anionic or amphoteric in nature, and combinations of the different types or combinations of the same type of surfactants may be used. Preferably, the surfactant will be amphoteric or nonionic in nature. Highly negative anionic surfactants are not very functional.

The last step is to dilute the salt-solvent-surfactant composition with water to the concentration desired for the selected dermatological medicament so as to form an emulsion, nano-emulsion or micro-emulsion depending on the micellar size and the choice of solvents/cosolvents.

### The Surfactants

For the purposes of this invention, it is preferred that the surfactants employed in the formation of emulsions, nano-emulsions or micro-emulsions of the salts of the invention are generally of the nonionic or amphoteric type or combinations of one or more nonionics, one or more amphoterics or one or more nonionics in combination with one or more amphoterics. Also, a cationic-amphoteric or cationic-nonionic surfactant system can be utilized to provide satisfactory results. Highly charged anionic surfactants are less desirable since they have the potential to reduce the bioactivity of the salts by causing some degree of precipitation and hence loss of cationic ions to the solution, thereby lessening the effectiveness of the salts.

It has also been found that cationic phospholipids, preferably in combination with nonionic and/or amphoteric surfactants are effective in the formation of micro-emulsions or emulsions of the salts of the invention.

Amphoteric surfactants that carry a positive charge in strongly acidic media carry a negative charge in strongly basic media, and form zwitterionic species at intermediate pH levels are amphoteric. Within the pH range of about 5.0 to 9.0 most amphoteric surfactants are primarily or fully in the zwitterionic (neutral) form, thereby negating any dilution of bioactivity due to reaction with the cationic portions of the dermatological salts. Amphoteric surfactants tend to be cationic at pHs below 5.0 and hence are also compatible with cationic ions from the dermatological salt in this pH range. The preferred concentration range for amphoteric surfactants is between about 0.25 and 6.0 wt. %, based on the weight of the salt of the invention in the final formulation.

The following surfactants have been found to be effective in the formation of emulsions, nano-emulsions and micro-emulsions of the salts of the invention: amphoteric amidobetaines; nonionic polyethoxylated sorbitol esters, polycondensates of ethylene oxide-propylene oxides (polyoxamers), polyethoxylated hydrogenated castor oils, and certain cationic phospholipids.

Suitable examples of amidobetaines include cocoamidoethyl betaine, cocoamido-propyl betaine; and mixtures thereof. Other suitable amphoteric surfactants include long chain imidazole derivatives such as the product marketed under the trade name “Miranol C2M” by Rhodia and long chain betaines such as the product marketed under the trade name “Empigen BB” by Huntsman Corporation, and mixtures thereof.

Suitable nonionic surfactants include polyethoxylated sorbitol esters, especially poly-ethoxylated sorbitol monoesters, e.g., PEG sorbitan diisostearate, and the products marketed under the trade name “Tween” by ICI; polycondensates of ethylene oxide and propylene oxide (polyoxamers), e.g., the products marketed under the trade name “Pluronic” by BASF; condensates of propylene glycol; polyethoxylated hydrogenated castor oil such as the products marketed under the trade name “Cremophors” by BASF; and sorbitan fatty esters marketed by ICI. Other effective nonionic surfactants include the polyalkyl(C8-C18) glucosides.

Suitable cationic surfactants include D,L-pyrrolidone-5-carboxylic acid salt of ethyl-cocoyl-L-arginate (CAE) marketed by Ajinomoto, and cocoamidopropyl (PTC), lauramidopropyl PG diammonium chloride phosphates and the like marketed by Uniqema. CAE and PTC have significant bioactivity and they therefore can be used as the cation of the binary cationic-anionic biocidal salts.

The choice of an effective surfactant system will differ somewhat for each biocidal salt of the invention. The choice of the surfactant system will depend upon the surfactant(s)' hydrophilic-lipophilic balance (HLB) to form a stable, small particle micelle in an aqueous or aqueous-cosolvent medium.

Other adjuvants useful in formulating the biocidal salts of the invention in o/w or w/o type creams, gels, lotions and the like include: polyether-modified silicone, cyclic silicone, methyl polysilicone, polyoxyethylene castor oil, cetostearyl alcohol, neopentyl glycol dicaprate, sorbitan monostearate, polyvinyl alcohol, glycerin, “Carbox”, glyceryl ether, cholesteryl isostearate, ethanol, isopropanol, glycerol monostearate PEG 100 stearate, hydroxymethyl cellulose, cetyl alcohol, lauryl glucoside and the like.

### Hydrophilic Polymers

A useful component of formulations of the invention is a water absorbing hydrophilic polymer with a molecular weight above 2000. Hydrophilic polymers act as skin substantive humectants and provide for moisture to be present on the skin. This moisture layer allows some of the controlled release salt to dissociate, releasing skin benefit agent. For this purpose, generally from about 0.05% to about 20% of hydrophilic polymer is required. Suitable hydrophilic polymers include polyacrylates, alginates, and cellulose derivatives such as methyl, hydroxyethyl and, hydroxypropyl cellulose. Also useful are gelatin, polydextrin, polyvinyl alcohol. Especially useful for this invention are hydrophilic copolymers known as “super slurpers” which are saponified starch-graft polyacrylonitrile copolymers. These are especially useful since small amounts of super slurper can hold larger amounts of moisture generally more than their own weight.

Especially useful hydrophilic polymers for increasing the moisture layer on skin are quarternized hydroxypolymers. Without being limited, specific examples include quaternary ammonium derivatives of hydroxyethyl cellulose, such as polyquaternium-4 and polyquaternium-10. Cationic guar gums are also effective, including, for example, guar hydroxypropyltrimonium chloride. These polymers not only retain moisture but are also highly effective skin conditioners. Many proteins and polypeptides with molecular weights above about 2000 are also useful for attracting and retaining moisture on the skin and in addition provide skin conditioning benefits.

Another group of useful polymers for attracting and retaining moisture on the skin are categorized as glycosaminoglycans and derivatives thereof. These polymers are natural or naturally derived. An important example of such a polymer is hyaluronic acid, which also provides skin lubricity.

### Skin Conditioners, Emollients, Emulsifiers

These following ingredients are acceptable if they do not react with the cationic antimicrobial agents and form a very water insoluble salt or significantly negatively affect the activity of the antimicrobial agent.

Skin conditioning agents/emollients include, but are not limited to, humectants, such a fructose, glucose, glycerin, propylene glycol, glycereth-26, mannitol, urea, pyrrolidone carboxylic acid, hydrolyzed lecithin, coco-betaine, cysteine hydrochloride, glucamine, PPG-15, sodium gluconate, potassium aspartate, oleyl betaine, thiamine hydrochloride, hydrolyzed proteins, hydrolyzed keratin, amino acids, amine oxides, water-soluble derivatives of vitamins A, E, and D, amino-functional silicones, ethoxylated glycerin, alpha-hydroxy acids and salts thereof, fatty oil derivatives, such as PEG-24 hydrogenated lanolin, and mixtures thereof. Numerous other skin conditioners are listed in the CTFA Cosmetic Ingredient Handbook, First Ed., J. Nikotakis, ed., The Cosmetic, Toiletry and Fragrance Association (1988), (hereafter CTFA Handbook), pages 79-84, incorporated herein by reference.

The skin conditioner/emollient also can be a water-insoluble ester having at least 10 carbon atoms, and preferably 10 to about 32 carbon atoms. Suitable esters include those comprising an aliphatic alcohol having about eight to about twenty carbon atoms and an aliphatic or aromatic carboxylic acid including from two to about twelve carbon atoms, or conversely, an aliphatic alcohol having two to about twelve carbon atoms with an aliphatic or aromatic carboxylic acid including about eight to about twenty carbon atoms. The ester is either straight-chained or branched. Suitable esters, therefore, include, for example, but are not limited to: (a) aliphatic monohydric alcohol esters, including, but not limited to: myristyl propionate, isopropyl isostearate, isopropyl myristate, isopropyl palmitate, cetyl acetate, cetyl propionate, cetyl stearate, isodecyl neopentanoate, cetyl octanoate, isocetyl stearate; (b) aliphatic di- and tri-esters of polycarboxylic acid, including, but not limited to: diisopropyl adipate, diisostearyl fumarate, dioctyl adipate, and triisostearyl citrate; (c) aliphatic polyhydric alcohol esters, including, but not limited to: propylene glycol dipelargonate; (d) aliphatic esters of aromatic acids, including, but not limited to: C12-C15 alcohol esters of benzoic acid, octyl salicylate, sucrose benzoate, and dioctyl phthalate. Numerous other esters are listed in the CTFA Handbook, at pages 24 through 26, incorporated herein by reference.

Sunscreen compounds as potential ingredients in the formulations of this invention are listed in CTFA Handbook, pages 86 and 87, incorporated herein by reference.

Other potential ingredients in the formulations of this invention include analgesics such as benzocaine, dyclonine hydrochloride, aloe vera, and the like; anesthetics such as butamben picrate, lidocaine hydrochloride, xylocaine, and the like; antiparasitics, such as lindane; essentially all dermatologicals, like acne preparations, such as benzoyl peroxide, erythromycin benzoyl peroxide, clindamycin phosphate, 5,7-dichloro-8-hydroxyquinoline, and the like; anti-inflammatory agents, such as alclometasone dipropionate, betamethasone valerate, and the like; burn relief ingredients, such as o-amino-p-toluenesulfonamide monoacetate, and the like; depigmenting agents, such as monobenzone; dermatitis relief agents, such as the active steroid amcinonide, diflorasone diacetate, hydrocortisone, and the like; diaper rash relief agents, such as methylbenzethonium chloride, and the like; emollients and moisturizers, such as mineral oil, PEG-4 dilaurate, lanolin oil, petrolatum, mineral wax, and the like; herpes treatment drugs, such as O-[(2-hydroxymethyl)-methyl]guanine; pruritic medications, such as alclometasone dipropionate, betamethasone valerate, isopropyl myristate MSD, and the like; psoriasis, seborrhea, and scabicide agents, such as anthralin, methoxsalen, coal tar, and the like; steroids, such as 2-(acetyloxy)-9-fluoro-1′,2′,3′,4′-tetrahydro-11-hydroxypregna-1,4-dieno-[16,17-b]naphthalene-3,20-dione and 21-chloro-9-fluoro-1′,2′,3′,4′-tetrahydro-11b-hydroxypregna -1,4-dieno-[16-,17-b]naphthalene-3,20-dione. Any other medication capable of topical administration, like skin protectants, such as allantoin, and antiacne agents, such as salicylic acid, also can be incorporated in a composition of the present invention in an amount sufficient to perform its intended function. Other topically applied compounds are listed in Remington's Pharmaceutical Sciences, 17th Ed., Mack Publishing Co., Easton, Pa. (1985), pages 773-791 and pages 1054-1058 (hereinafter Remington's), incorporated herein by reference.

Also topically active compounds such as a plant extract or a natural oil can be added to the formulations of this invention. Numerous plant extracts are available from Lipo Chemicals, Inc. Paterson, N.J. Nonlimiting plant extracts are those obtained from alfalfa, aloe vera, amla fruit, angelica root, anise seed, apple, apricot, artichoke leaf, asparagus root, banana, barberry, barley sprout, bee pollen, beet leaf, bilberry fruit, birch leaf, bitter melon, black currant leaf, black pepper, black walnut, blueberry, blackberry, burdock, carrot, cayenne, celery seed, cherry, chickwood, cola nut, corn silk, cranberry, dandelion root, elderberry, eucalyptus leaf, flax oil powder, ginger root, gingko leaf, ginseng, goldenrod, goldenseal, grape, grapefruit, guava, hibiscus, juniper, kiwi, kudzu, lemon, licorice root, lime, malt, marigold, myrrh, olive leaf, orange fruit, orange peel, oregano, papaya fruit, papaya leaf, passion fruit, peach, pear, pine bark, plum, pomegranate, prune, raspberry, rhubarb root, rosemary leaf, sage leaf, spearmint leaf, St. John's wart, strawberry, sweet cloves, tangerine, violet herb, watercress, watermelon, willow bark, wintergreen leaf, witch hazel bark, yohimbe, and yucca root. An example of a natural oil is rice bran oil.

The formulations of this invention also can contain a hydrotrope. A hydrotrope is a compound that has an ability to enhance the water solubility of other compounds. Specific examples of hydrotropes include, but are not limited to, sodium cumene sulfonate, ammonium cumene sulfonate, ammonium xylene sulfonate, potassium toluene sulfonate, sodium toluene sulfonate, sodium xylene sulfonate, toluene sulfonic acid, and xylene sulfonic acid. Other useful hydrotropes include sodium polynaphthalene sulfonate, sodium polystyrene sulfonate, sodium methyl naphthalene sulfonate, sodium camphor sulfonate, and disodium succinate.

The formulations of this invention also can contain an additional organic solvent. The solvent can be a water-soluble organic compound containing one to six, and typically one to three, hydroxyl groups, e.g., alcohols, diols, triols, and polyols. Specific examples of solvents include, but are not limited to, methanol, ethanol, isopropyl alcohol, n-butanol, n-propyl alcohol, ethylene glycol, propylene glycol, glycerol, diethylene glycol, dipropylene glycol, tripropylene glycol, hexylene glycol, butylene glycol, 1,2,6-hexanetriol, sorbitol, PEG-4, 1,5-pentanediol, similar hydroxyl-containing compounds, and mixtures thereof. The solvent also can be water or an aprotic solvent, e.g., dimethyl sulfoxide or tetrahydrofuran.

The formulations of this invention also can contain a thickening or gelling agent. A thickening or gelling agent can be, for example, a polymer that is water soluble or that generates a colloidal solution in water. A thickening or gelling agent, therefore, can be, for example, polymers or copolymers unsaturated carboxylic acids or unsaturated esters, polysaccharide derivatives, gums, colloidal silicates, polyethylene glycols (PEG) and their derivatives, polyvinylpyrrolidones and their derivatives, polyacrylamides and their derivatives, polyacrylonitriles, hydrophilic silica gels, or mixtures thereof.

Specific thickening or gelling agents can be, for example, acrylic and/or methacrylic polymers or copolymers, vinylcarboxylic polymers, polyglyceryl acrylates or methacrylates, polyacrylamides derivatives, cellulose or starch derivatives, chitin derivatives, alginates, hyaluronic acid and its salts, chonodroitin sulphates, xanthan, gellan, Rhamsan, karaya or guar gum, carob flour, and colloidal aluminum magnesium silicates of the montmorillonite type.

Additional thickening or gelling agents include vinylcarboxylic polymers sold under the tradename CARBOPOL® (Goodrich), acrylic acid/ethyl acrylate copolymers, acrylic acid/stearyl methacrylate copolymers, carboxymethylcellulose, hydroxymethylcellulose, hydroxypropylcellulose, microcrystalline cellulose, hydroxypropyl guar, colloidal hectorites, bentonites, and the like.

Other classes of optional ingredients for formulations of this invention included in a present composition can be, but not limited to, pH adjusters, chelating agents, preservatives, buffering agents, foam stabilizers, opacifiers, and similar classes of ingredients known to persons skilled in the art. Specific optional ingredients include inorganic phosphates, sulfates, and carbonates as buffering agents; EDTA and phosphates as chelating agents; and acids and bases as pH adjusters.

Non-limiting examples of basic pH adjusters are ammonia; mono-, di-, and tri-alkyl amines; mono-, di-, and tri-alkanolamines; alkali metal and alkaline earth metal hydroxides; and mixtures thereof. Specific, nonlimiting examples of basic pH adjusters are ammonia; sodium, potassium, and lithium hydroxide; monoethanolamine; triethylamine; isopropanolamine; diethanolamine; and triethanolamine. Examples of acidic pH adjusters/buffers are the mineral acids and organic carboxylic acids. Nonlimiting examples of mineral acids are citric acid, hydrochloric acid, nitric acid, phosphoric acid, and sulfuric acid.

Any conventionally usable emulsifier can be used in the formulations of this invention. Emulsifier systems may comprise for example:

Carboxylic acids and their salts: alkaline soap of sodium, potassium and ammonium, metallic soap of calcium or magnesium, organic basis soap such as Lauric, palmitic, stearic and oleic acid and the like

Alkyl phosphates or phosphoric acid esters: acid phosphate, diethanolamine phosphate, potassium cetyl phosphate.

Ethoxylated Carboxylic Acids or Polyethyleneglycol esters (PEG-n Acylates). Linear fatty alcohols having from 8 to 22 carbon atoms, branched from 2 to 30 mol of ethylene oxide and/or from 0 to 5 mol propylene oxide with fatty acids having from 12 to 22 carbon atoms and with alkylphenols having from 8 to 15 carbon atoms in the alkyl group. Fatty alcohol Polyglycol ether such as Laureth-n, Ceteareth-n, Steareth-n, Oleth-n. Fatty acid polyglycolether such as PEG-n Stearate, PEG-n Oleate, PEG-n Cocoate, monoglycerides and Polyol esters. C12-C22 fatty acid mono- and di-esters of addition products of from 1 to 30 mol of ethylene oxide with polyols, Fatty acid and polyglycerol ester such as Monostearate glycerol, diisostearoyl polyglyceryl-3-diisostearates, polyglyceryl-3-diisostearates, triglyceryl diisostearates, polyglyceryl-2-sesquiisostearates or polyglyceryl dimerates. Mixtures of compounds from a plurality of those substance classes are also suitable. Fatty acid polyglycolesters such as Monostearate diethylene glycol, Fatty acid and Polyethylene glycol esters, Fatty acid and saccharose esters such as Sucro esters, glycerol and saccharose esters such as Sucro glycerides

Sorbitol and sorbitan: Sorbitan mono- and di-esters of saturated and unsaturated fatty acids having from 6 to 22 carbon atoms and ethylene oxide addition products

Polysorbate-n series, Sorbitan esters such as Sesquiisostearate, sorbitan, PEG-(6)-isostearate sorbitan, PEG-(10)-Sorbitan laurate, PEG-17-dioleate sorbitan.

Glucose derivatives: C₈-C₂₂ alkyl-mono and oligo-glycosides and ethoxylated analogues with glucose being preferred as the sugar component. O/W emulsifiers such as Methyl Gluceth-20sesquistearate, Sorbitan Stearate/Sucrose Cocoate, Methyl Glucose Sesquistearate, Cetearyl alcohol/Cetearyl glucoside. W/O emulsifiers such as Methyl glucose Dioleate/Methyl glucose isostearate.

The following sulfates and sulfonated derivatives are acceptable if they do not react with the cationic antimicrobial agents and form a very water insoluble salt or significantly negatively affect the activity of the antimicrobial agent: Dialkylsulfosuccinates (DOSS: Dioctyl succinate), alkyl lauryl sulfonate, linear sulfonated parafins, sulfonated tetrapropylene sulfonate, sodium lauryl sulfates, ammonium and ethanolamine lauryl sulfates, lauryl ether sulfates, sodium laureth sulfates, sulfosuccinates, acetyl isothionates, alkanolamide sulfates such as Taurines, Methyl taurines, Imidazole sulfates

Amine derivatives: amine salts, ethoxylated amines such as Oxide amine, with chains containing an heterocycle such as alkyl imidazolines, pyridine derivatives, isoquinoteines, cetyl pyridinium chloride, cetyl pyridinium bromide, quaternary ammonium such as cetyltrimethyl ammonium bromide (CTBA), Stearylalkonium.

Amide derivatives: alkanolamides such as acylamide DEA, ethoxylated amides, such as PEG-n acylamide, oxydeamide.

Polysiloxane/Polyalkyl/Polyether Copolymers and derivatives: dimethicone, copolyols, silicone polyethylene Oxide copolymer, silicone glycol copolymer.

Propoxylated or POE-n ethers (Meroxapols), Polaxamers or poly(oxyethylene)m-block-poly(oxypropylene)n-block(oxyethylene)

Zwitterionic surfactants that carry at least one quaternary ammonium group and at least one carboxylate and/or sulfonate group in the molecule. Zwitterionic surfactants that are especially suitable are the so-called betaines, such as N-alkyl-N,N-dimethylammonium glycinates, for example cocoalkyidimethylammonium glycinate, N-acylaminopropyl-N,N-dimethylammonium glycinates, for example cocoacylaminopropyidimethylammonium glycinate, and 2-alkyl-3-carboxymethyl-3-hydroxyethylimidazolines each having from 8 to 18 carbon atoms in the alkyl or acyl group and also cocoacylaminoethylhydroxyethyl-carboxy-methylglycinate, N-alkylbetaine, N-alkylaminobetaines.

Alkylimidazolines, alkylopeptides, lipoaminoacides.

Self-emulsifying bases (K. F. DePolo—A short textbook of cosmetology, Chapter 8, Table 8-7, p 250-251):

Nonionic bases such as PEG-6 Beeswax (and) PEG-6 Stearate (and) polyglyceryl-2 isostearate [Apifac], Glyceryl stearate (and) PEG-100 stearate. [Arlacel 165], PEG-5 Glyceryl stearate [arlatone 983 S], Sorbitan oleate (and) Polyglyceryl-3 Ricinoleate [Arlacel 1689], Sorbitan Stearate and sucrose cocoate [arlatone 2121], Glyceryl stearate and laureth-23 [Cerasynth 945], Cetearyl alcohol and ceteth-20 [Cetomacrogol Wax], Cetearyl alcohol and Polysorbate 60 and PEG-150 and stearate-20 [Polawax GP 200, Polawax NF], Cetearyl alcohol and cetearyl polyglucoside [Emulgade PL 1618], Cetearyl alcohol and ceteareth-20 [Emulgade 1000NI, Cosmowax], Cetearyl alcohol and PEG-40 castor oil [Emulgade F Special], Cetearyl Alcohol and PEG-40 Castor Oil and Sodium Cetearyl Sulfate [Emulgade F], Stearyl Alcohol and Steareth-7 and Steareth-10 [Emulgator E 2155], Cetearyl Alcohol and Szeareth-7 and steareth-10 [Emulsifying wax U.S.N.F], Glyceryl stearate and PEG-75 stearate [Gelot 64], Propylene Glycol ceteth-3 Acetate [Hetester PCS], Propylene Glycol isoceth-3 Acetate [Hetester PHA], Cetearyl alcohol and Ceteth-12 and oleth-12 [Lanbritol Wax N 21], PEG-6 Stearate and PEG-32 Stearate [Tefose 1500], PEG-6 Stearate and ceteth-20 and steareth-20 [Tefose 2000], PEG-6 Stearate and ceteth-20 and Glyceryl Stearate and steareth-20 [Tefose 2561], Glyceryl Stearate and ceteareth-20 [Teginacid H, C₁ X].

Nonionic substances such as PEG-2 Stearate SE, Glyceryl stearate SE [Monelgine, Cutina K D], Propylene glycol stearate [Tegin P].

Nonionic substances such as cetearyl alcohol, cetearyl alcohol and glycol stearate and PEG-2 Stearate [Sedefos 75], Glyceryl stearate

Cationic acid bases such as cetearyl alcohol and cetrimonium bromide.

When formulated in O/W emulsions, the preferable amount of such emulsifier system could represent 5% to 20% of the oil phase.

### Applications of the Salts of the Invention

Set forth below is a representative list of some of the numerous possible applications of the salts of the present invention. It is to be understood that this list is presented for illustrative purposes only and should not be construed as representing any limitation as to possible applications of the salts. It is to be further understood that it is within the purview of the invention to combine the salts with other ingredients such as conventional antioxidants, antibacterial agents, antifungal agents, hormones, vitamins, hydroxy acids, cleansers, soaps, shampoos, silicones, biocides, humectants, emollients, synthetic and/or natural oils, deodorizers, perfumes, colorants, preservatives, plant extracts, analgesics, antiparasitic agents, anti-pigmenting argents, anti-inflammatory agents, diaper rash relief agents, fungicides, herpes treatment drugs, pruritic medications, psoriasis, seborrhea, and scabicide agents, steroids, pH adjusters/buffers, and the like. The ingredients are included in the formulations of this invention in an amount sufficient to perform their intended function.

Mammalian skin/nail care products, e.g., sunscreens, suntan lotions, after-sun lotions, gels creams and sprays; antiperspirants; deodorants (liquids, powders, gels, roll-ons, sticks, sprays, pastes, creams, lotions); cleansing creams; skin conditioners; skin moisturizers; protectants; skin aging products; skin wrinkle-reduction products; acne treatment products; rosacea treatment products; age-spot reduction products; stretch-mark reduction products; pimple treatment products; skin soothing products; skin infection and lesion treatment products; skin-redness reduction products; varicose- and spider-vein reduction products; lotions; oils; hand/body creams; shaving gels, foams and creams; body washes; liquid and solid soap products; blood microcirculation improvement products; cellulite-reduction products; body toning products; skin penetration enhancers; skin whitening products; cosmetics; medicinal shampoos; shower gels; bubble baths; nail treatment products; hand (or mechanical) dishwashing products; hand sanitizers and disinfectants; lipsticks and lip balms; salves; collodion; impregnated patches and strips for skin treatment; skin surface implants; impregnated or coated diapers; cosmetic basecoats and undercoats, bath capsules, bath oils, bath tablets, bath salts, bath soaps, blushers, face, body, and hand creams and lotions, cosmetic foundations, hormone creams and lotions, leg and body paints, makeup bases, makeup fixatives, makeup products, moisturizing creams and lotions, night creams and lotions, paste masks, skin care products, skin fresheners, skin lighteners, tonics, dressings, and the like. As noted previously, in a body wash or other product where the salts of this invention come in contact with the body when there is an abundance of water, e.g. a body wash, etc., the solubility of the salts of this invention needs to be lower than if the salts of the invention are in a cream or ointment that does not normally come into contact with copies amounts of water, e.g. an antifungal foot treatment, etc.

The following examples shall serve to illustrate the various embodiments of the invention. The examples are given solely for the purpose of illustration and are not to be construed as limitations of the present invention since many variations are possible without departing from the spirit and scope of the invention. Unless otherwise indicated, all parts and percentages are on a weight basis.

### Example 1

This example pertains to an acne treatment wash formulation containing a controlled release chlorhexidine-salicylate salt. The benefit is that the salt will slowly release the ions in the presence of skin moisture and also will be neutral until the anion is protonated by the acidity of the skin, thereby not burning the skin. It will also allow the formulation to be more neutral instead of requiring a low pH for the formulation to stabilize the salicylic acid that is normally used as the active in an acne treatment.

| Ingredient | Weight % |
|---|---|
| Chlorhexidine-salicylate salt | 0.5 |
| “Tego Betaine ZF” | 1.5 |
| Propylene Glycol | 15.0 |
| Propylene Glycol Dicaprylate-Dicaprate | 8.0 |
| Hydroxyethyl Cellulose | 0.25 |
| PEG 40 Stearate | 2.50 |
| “Stearath-2” | 1.00 |
| Water | QS |

### Example 2

This example pertains to an after shower spray containing N^{α}-lauroyl-L-arginine-ethyl ester (“LAE”) -lactate salt. The ethanol acts as a quick drying ingredient and also acts as a deodorizer, however after it evaporates, bacteria can grow back. LAE-HCl is green and naturally derived and has antimicrobial properties. It is used as a preservative in ready to eat foods and in cosmetics; however, it is very water soluble and reacts with anions normally found in cosmetic formulas. When made into the LAE-lactate sat, it has lower water solubility and has controlled release properties. Lactic acid is a type of alpha hydroxy acid (AHA) and also is an antimicrobial at higher concentrations than are used in this formulation, e.g. about 3% is used in household cleaners. Lactic acid is a powerful exfoliant when used at about 10% in some natural skin care formulas and is featured in many skin care products. Just like glycolic acid, another popular AHA, lactic acid is very effective in reducing fine lines, wrinkles, age spots and hyperpigmentation at higher concentrations. Moreover, it also enhances the skin texture and stimulates the production of collagen within the skin cells. Lactic acid alone has a noticeable odor, but in making a lactate salt, it mostly eliminates the odor. The LAE-lactate salt acts as an emollient with antimicrobial and skin benefit characteristics and will release its ions slowly based on the moisture on the skin to give longer term deodorancy (as compared to the fast acting ethanol) as well as an exfoliating benefit. The LAE-lactate salt thus has dual functionality.

| Ingredient | Weight % |
|---|---|
| LAE-lactate salt | 1.00 |
| Ethanol | 50.00 |
| Hydroxypropyl Cellulose | 0.50 |
| “Tego Betaine ZF” | 2.00 |
| 1,3-Propylene Glycol | 10.00 |
| Dye | 0.020 |
| DI Water | QS |

### Example 3

This example pertains to a body oil formulation containing a benzalkonium laurate salt. The presence of the controlled release benzalkonium-laurate salt in this formulation provides extended deodorancy benefit from both ions to the skin.

| Ingredient | Weight % |
|---|---|
| Benzalkonium laurate | 0.1 |
| Polysorbate 60 | 3.00 |
| Sorbitan stearate | 2.00 |
| Cetyl alcohol | 0.75 |
| Paraffin Wax | 3.00 |
| Isopropyl myristate | 2.50 |
| Caprylic-caproic triglycerides | 2.00 |
| Dimethicone | 0.50 |
| 1,3-Propylene glycol | 2.00 |
| DI water | QS |

### Example 4

This example pertains to a face masque/cleaner formulation containing a LAE-salicylate salt. The presence of the controlled release salt LAE-salicylate in this formulation provides exfoliant benefits from the salicylate anions to the skin. The benefits of neutral pH and less skin burning pertaining to the use of salicylate anions instead of salicylic acid are similar to those as listed in example #1.

| Ingredient | Weight % |
|---|---|
| LAE-salicylate | 0.15 |
| Stearic acid | 2.00 |
| Glyceryl stearate | 2.50 |
| Cetyl alcohol | 1.50 |
| Paraffin wax | 5.00 |
| Isopropyl myristate | 1.50 |
| Caprylic-caproic triglycerides | 1.00 |
| Dimethicone | 1.50 |
| 1,3-Propylene glycol | 2.00 |
| Triethanolamine | to pH of 5.2 |
| DI Water | QS |

### Example 5

This example pertains to an athlete's foot gel treatment formulation containing a controlled release LAE-undecylenate salt. The benefit is that the salt will slowly release the ions in the presence of skin moisture and also will be more adherent to the skin and less water soluble than the LAE-HCl salt. The ethanol will act as a quick antimicrobial to rapidly kill germs. The LAE-undecylenate controlled release salt is an emollient with anti-fungal characteristics and will act as an “inactive enabler” to give extended activity and improved efficacy to the alcohol formula while not being an active antimicrobial in itself. The LAE-undecylenate salt thus has dual functionality.

| Ingredient | Weight % |
|---|---|
| LAE-undecylenate | 0.2 |
| Ethanol | 65.0 |
| Carbomer | 0.5 |
| Glyceryl caprylate | 0.2 |
| Cocamidopropyl betaine | 0.5 |
| Triethanolamine | To pH 5.5 |
| Water | 33.6 |

### Example 6

This example pertains to a Jock Itch Infection Ointment treatment formulation containing a controlled release Benzalkonium-hexanoate salt. The Benzalkonium chloride will act as a quick antimicrobial to rapidly kill germs. The benefit is that the Benzalkonium-hexanoate salt will slowly release the ions in the presence of skin moisture after the Benzalkonium chloride active ingredient is depleted, allowing for a longer period of activity and longer relief from itching.

| Ingredient | Weight % |
|---|---|
| Benzalkonium hexanoate | 0.25 |
| Benzalkonium chloride | 0.05 |
| Carboxymethyl cellulose | 2.00 |
| Carbowax 3400 | 25.00 |
| Carbowax 600 | 72.70 |

### Example 7

This example pertains to a Deodorizing Body Powder formulation containing a controlled release Cetyl pyridinium Octanoate salt. The benefit is that the Cetyl pyridinium Octanoate salt will slowly release the ions in the presence of skin moisture, providing for a longer period of deodorancy than if Cetyl pyridinium chloride was the active ingredient. Because the Cetyl pyridinium Octanoate controlled release salt is less soluble than Cetyl pyridinium chloride, the salt will remain on the body longer because the salt will not be depleted by the skin perspiration as quickly as the chloride salt.

| Ingredient | Weight % |
|---|---|
| Talc (Asbestos-free) | 50.0 |
| Corn Starch | 47.5 |
| Cetyl pyridinium Octanoate | 0.5 |
| Polyquaterrnium-10 | 2.0 |

### Example 8

This example pertains to a Lip Balm for Herpes formulation containing a controlled release LAE-Decanoate salt. The benefit is that the LAE-Decanoate salt will slowly release the ions in the presence of skin moisture, providing for a longer period of activity. LAE-decanoate is an emollient with preservative characteristics. Because the LAE-Decanoate controlled release salt has low solubility, it can be formulated in a lip balm. Since decanoic acid is a fatty acid, forming a salt or ester with LAE will increase its lipophilicity and its affinity for fatty tissue. The green and naturally derived LAE salt with decanoic acid decanoic acid is found in coconut milk) will aid in moisturizing and soothing the skin and will provide a pleasant feeling.

| Ingredient | Weight % |
|---|---|
| LAE-Decanoate | 0.3 |
| Lanolin Oil | 8.0 |
| Bleached Beeswax | 35.0 |
| Mineral Oil | 27.0 |
| Polyglyceryl-4 monooleate | 1.0 |
| Paraffin wax | 14.0 |
| Polysorbate 20 | 2.0 |
| Vitamin E Acetate | 0.5 |
| DI Water | 10.2 |
| Carboxymethyl cellulose | 2.0 |

1. A method comprising the application of a formulation to the skin or nails comprising from about 0.02 wt % to 5 wt % of a salt of a monomeric or polymeric cation and a monomeric or polymeric carboxylate anion and at least one or more of the following: (1) from about 0.05% to about 20 wt % of a water absorbing hydrophilic polymer with a molecular weight above 2000, (2) from about 1% to about 90 wt % of an anionic, nonionic or amphoteric surfactant or soap, (3) from about 0.02 to about 25 wt % of an emollient (4) from about 0.02 to about 99 wt % of an emulsifier, (5) buffers to provide a pH between about 3.0 and 7.0, (6) L-cysteine or L-N-acetyl cysteine, or (7) from about 2% to about 95 wt % moisture.

2. The method of claim 1 wherein the cation comprises a cation selected from the group consisting of a cation of an amidine, a cation of a guanidine, a cation of a biguanide, a quaternary ammonium cation, a cation of an azole with an amine functional group, a cation of an antibiotic with an amine functional group, and a cation of a dibasic amino acid.

3. The method of claim 2 wherein the dibasic amino acid is selected from the group consisting of arginine, histidine, lysine and ornithine.

4. The method of claim 2 where the cation of a guanidine is selected from N^{α}-lauroyl-L-arginine ethyl ester and polyhexamethylene guanidine.

5. The method of claim 1 wherein the cation is selected from the group consisting of N^{α}-lauroyl-L-arginine ethyl ester, N^{α}-lauroyl-L-histidine ethyl ester and N^{α}-lauroyl-L-lysine ethyl ester.

6. The method of claim 2 wherein the biguanide cation is selected from the group consisting of cations of chlorhexidine, hexetidine, alexidine, and polyhexamethylene biguanide.

7. The method of claim 1 wherein the carboxylate anion contains a saturated or unsaturated functional group selected from the group consisting of aliphatic, aromatic and alicyclic groups.

8. The method of claim 1 wherein the carboxylate anion is selected from the group consisting of a monobasic aliphatic carboxylate, a monobasic aromatic carboxylate, and a monobasic alicyclic carboxylate.

9. The method of claim 8 wherein the monobasic aliphatic carboxylate anion is selected from the group consisting of the anion of lauric acid, the anion of palmitic acid, the anion of myristic acid, the anion of oleic acid, the anion of stearic acid, the anion of dehydroacetic acid and the anion of undecylenic acid.

10. The method of claim 8 wherein the monobasic aliphatic carboxylate contains a hydroxyl group or a ketone group.

11. The method of claim 8 wherein the monobasic aliphatic carboxylate is selected from the group consisting of the anion of glycolic acid, the anion of gluconic acid, the anion of glyceric acid and the anion of lactic acid.

12. The method of claim 8 wherein the monobasic aromatic carboxylate anion is the anion of salicylic acid.

13. The method of claim 1 wherein the carboxylate anion is derived from a carboxylic acid molecule containing at least two carboxylic acid groups.

14. The method of claim 13 wherein the carboxylate anion is selected from the group consisting of the anion of citric acid, the anion of malic acid, the anion of tartaric acid and the anion of azelaic acid, the anion of glutaric acid, the anion of glutamic acid, and the anion of their derivatives.

15. The method of claim 1 wherein the salt has a maximum solubility in aqueous media of about 5 wt. %.

16. The method of claim 15 wherein the salt has a maximum solubility in aqueous media of 2 wt. %.

17. The method of claim 15, wherein the solubility is greater than 0.01 wt.%.

18. The method of claim 1 wherein the salt is selected from the group consisting of the laurate of N^{α}-lauroyl-L-arginine ethyl ester, the salicylate of N^{α}-lauroyl-L-arginine ethyl ester, the lactate of N^{α}-lauroyl-L-arginine ethyl ester, the citrate of N^{α}-lauroyl-L-arginine ethyl ester, the malate of N^{α}-lauroyl-L-arginine ethyl ester, the gluconate of N^{α}-lauroyl-L-arginine ethyl ester, the azelate of N^{α}-lauroyl-L-arginine ethyl ester, the glycolate of N^{α}-lauroyl-L-arginine ethyl ester, the glycerate of N^{α}-lauroyl-L-arginine ethyl ester, the hyaluronate of N^{α}-lauroyl-L-arginine ethyl ester, the arachidonate of N^{α}-lauroyl-L-arginine ethyl ester, the oleate of N^{α}-lauroyl-L-arginine ethyl ester (C₁₈, unsaturated), the linoleate of N^{α}-lauroyl-L-arginine ethyl ester (C₁₈, polyunsaturated), the a-linoleate of N^{α}-lauroyl-L-arginine ethyl ester acid (ALA), the eicosapentaenoate of N^{α}-lauroyl-L-arginine ethyl ester acid (EPA), the docosahexaeonate of N^{α}-lauroyl-L-arginine ethyl ester (DHA), the erucate of N^{α}-lauroyl-L-arginine ethyl ester, the tartrate of N^{α}-lauroyl-L-arginine ethyl ester and the 3-hydroxypropionate of N^{α}-lauroyl-L-arginine ethyl ester.

19. A formulation for treating skin or nails comprising from about 0.02 wt % to 5 wt % of a salt of a monomeric or polymeric cation and a monomeric or polymeric carboxylate anion and at least one or more of the following: (1) from about 0.05% to about 20 wt % of a water absorbing hydrophilic polymer with a molecular weight above 2000, (2) from about 1% to about 90 wt % of an anionic, nonionic or amphoteric surfactant or soap, (3) from about 0.02 to about 25 wt % of an emollient (4) from about 0.02 to about 99 wt % of an emulsifier, (5) buffers to provide a pH between about 3.0 and 7.0, (6) L-cysteine or L-N-acetyl cysteine, and (7) from about 2% to about 95 wt % moisture.

20. The formulation of claim 20, wherein the salt has a solubility of less than 5 wt. % to greater than about 0.01 wt. %.
